(19) United States
(12) Patent Application Publication
(10) Pub. No.: US 2005/0273913 A1
(43) Pub. Date: Dec. 15, 2005

(54) Swimming goggles

(76) Inventor: Herman Chiang, Chung-Ho City (TW)

Correspondence Address: TROXELL LAW OFFICE PLLC, SUITE 1404, 5205 LEESBURG PIKE, FALLS CHURCH, VA, 22041, US

(21) Appl. No.: 10/852,194

(22) Filed: May 25, 2004

(51) Int. Cl. A61F 9/02

(52) U.S. Cl. 2/428

(57) ABSTRACT

Swimming goggles have a left frame, a right frame, head strap, a nose support connecting the left frame and the right frame. Lenses are slantwise formed in the left frame and right frame. The width between upper edges of inner surface of the lenses and upper edges of the left and the right frames is smaller than the width between lower edges of inner surface of the lenses and lower edges of the left and the right. In use, the lenses fit to the user's face and eyes for maintaining almost horizontal relative to the eyes and close to the eyes, thereby reducing resistance force when a user jumps into water and enlarging visual field of the user.

### BACKGROUND OF THE INVENTION

1. Field of the Invention

The present invention relates to swimming goggles, and particularly to swimming goggles which reduce resistance force when jumping into water and which expand visual field when swimming.

2. Related Art

A variety of prior art swimming goggles commonly enclose around periphery of users' eyehole. Such swimming goggles project beyond a user's face so much that resistance force is considerable. Correspondingly, the swimming goggles tend to move, resulting in water leakage.

Moreover, because upper edge of user's eyehole is slightly convex while lower edge is slightly concave, the upper and lower edges of the left and right frames of the swimming goggles are often inclined with respect to the user's eyes. Accordingly, upper edges of lenses of the swimming goggles are farther from eyes, making visual field smaller.

### SUMMARY OF THE INVENTION

Accordingly, an object of the present invention is to provide swimming goggles which reduce resistance force when jumping into water and which expand visual field when swimming.

Another object of the present invention is to provide lenses of the swimming goggles which remain alignment with a user's eyes almost horizontal after in use.

The swimming goggles comprise a left frame, a right frame, head strap, and a nose support connecting the left frame and the right frame. Lenses are unitarily formed with the left frame and the right frame. An enveloping frames are respectively formed on the left frame and the right frame and are close to a user's face. The width between upper edges of inner surface of the lenses and upper edges of the enveloping frames is smaller than the width between lower edges of inner surface of the lenses and lower edges of the enveloping frames

The lenses are slantwise arranged in the left and the right frames such that in assembly, the width between upper edges of inner surface of the lenses and upper edges of the left and the right frames is smaller than the width between lower edges of inner surface of the lenses and lower edges of the left and the right. Therefore, the lenses fit to the user's eyes for maintaining almost horizontal relative to the eyes.

### BRIEF DESCRIPTION OF THE DRAWINGS

FIG. 1 is an exploded view of swimming goggles of the present invention.

FIG. 2 is an assembled view of the swimming goggles of FIG. 1.

FIG. 3 is a front view of the swimming goggles of FIG. 2.

FIG. 4 is a cross-sectional view taken along the line 4-4 in FIG. 3.

FIG. 5 is a cross-sectional view taken along the line 5-5 in FIG. 3.

FIG. 6 is a cross-sectional view taken along the line 6-6 in FIG. 3.

FIG. 7 is a cross-sectional view taken along the line 7-7 in FIG. 3.

### DESCRIPTION OF THE PREFERRED EMBODIMENTS

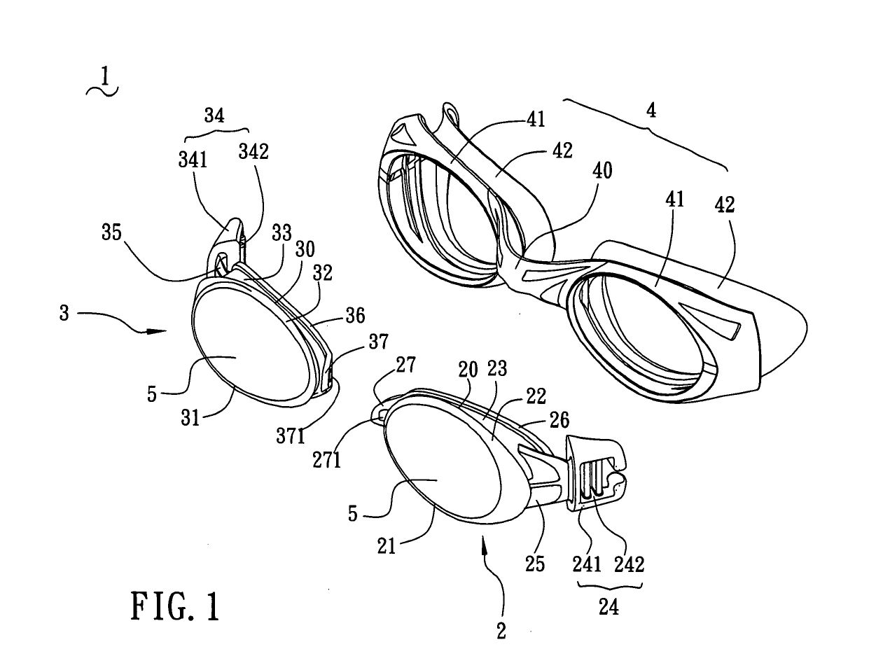

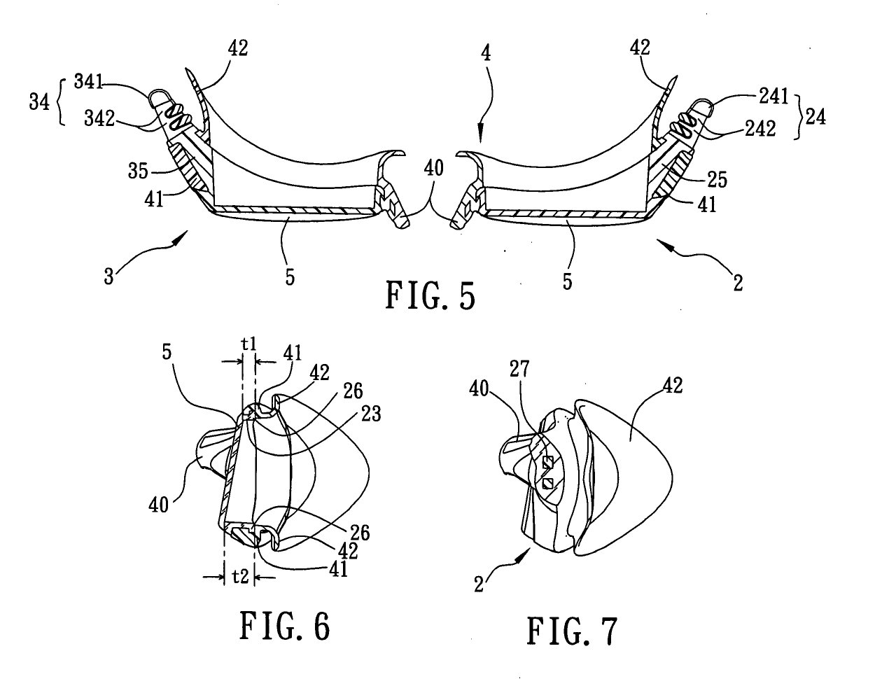

With reference to FIG. 1, swimming goggles 1 of the present invention comprise a left frame 2, a right frame 3, a connecting frame 4 connecting the left frame 2 and the right frame 3 and a head strap (not shown). The left frame 2 and the right frame 3 are made of hard material and respectively have inner surfaces 20, 30, outer surfaces 21, 31 and peripheral surfaces 22, 32 between the outer surfaces 21, 31 and the inner surfaces 20, 30. Enveloping frames 23, 33 are respectively formed around the peripheral surfaces 22, 32 and slightly offset relative to outer peripheries of the inner surfaces 20, 30. Fastener bases 24, 34 are respectively formed on outward sides of the enveloping frames 23, 33. Further referring to FIGS. 4 and 5, the fastener bases 24, 34 include bodies 241, 341, and connecting portions 25, 35 respectively for connecting the fastener bases 24, 34 with the left frame 2 and the right frame 3. The connecting portions 25, 35 space the bases 241, 341 a distance from the inner surfaces 20, 30, thereby providing more space for facilitating the connecting frame 4 to support the enveloping frames 23, 33 of the left frame 2 and the right frame 3. Projections 26, 36 are formed along outer peripheries of the enveloping frames 23, 33 for providing more space for the connecting frame 4 as well. Fastener holes 242, 342 are respectively defined in the bodies 241, 341 for movably receiving head strap. Lenses 5 are integrally formed with the outer surfaces 21, 31. As shown in FIG. 6, the width to between upper edges of inner surface of the lenses 5 and upper edges of the projections 26 is smaller than the width t2 between lower edges of inner surface of the lenses 5 and lower edges of the projections 26. Showed in embodiment, the width t1 between upper edges of inner surface of the lenses 5 and upper edges of the enveloping frames 23, 33 meet along upper edge of inside of user's eyehole, and the width t2 between lower edges of inner surface of the lenses 5 and lower edges of the enveloping frames 23, 33 meet along lower edges of inside of user's eyehole. Thus, in use, the lenses 5 can remain alignment with and closer to the user's eyes. Support portions 27, 37 integrally and inwardly extend from the enveloping frames 23, 33. Through holes 271, 371 are respectively defined in the support portions 27, 37 for facilitating the connecting frame 4 to support the left frame 2 and the right frame 3.

The connecting frame 4 is made of soft material, and includes a nose support 40 for enclosing the support portions 27, 37, a linking portion 41 for enclosing the enveloping frames 23, 33, and pads 42 connecting with the linking portion 41 for providing comfortable touch for the user.

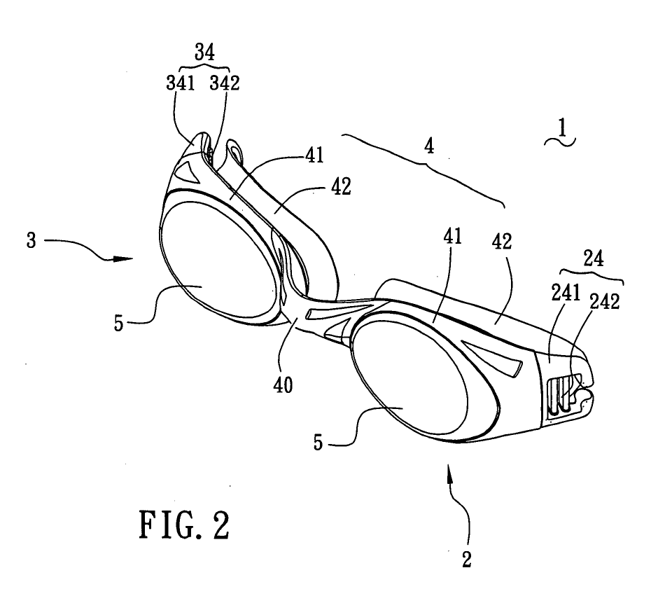

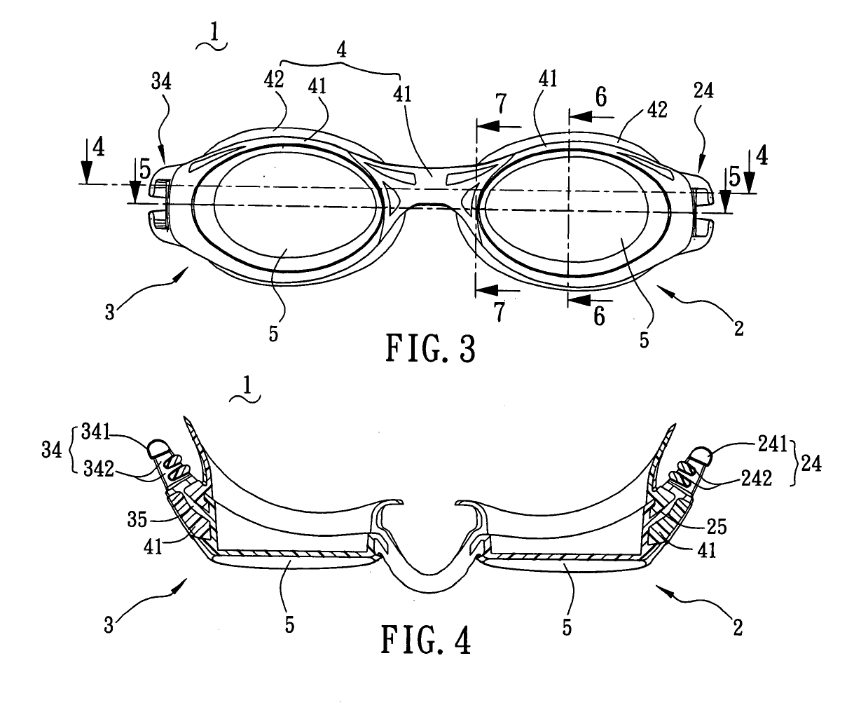

With reference to FIGS. 2 and 3, in assembly, the connecting frame 4 unitarily connects the left frame 2 and the right frame 3 together. Further referring to FIGS. 1 and 7, the nose support 40 surrounds the support portions 27, 37. As shown in FIG. 6, the connecting frame 4 encircles the enveloping frames 23, 33, thereby supporting the left frame 2 and the right frame 3 reliably. Referring to FIGS. 3 and 4, the connecting portions 25, 35 produce offset between the fastener bases 24, 34 and the left frame 2 and the right frame 3, whereby the connecting frame 4 encloses outer peripherals of the left frame 2 and the right frame 3 firmly. In other words, the nose support 40, the link portions 41 and the pads 42 made of soft material enclose the left frame 2 and the right frame 3 made of hard material. Thus head strap can be easily pulled through fastener holes 242, 342 of the left frame 2 and the right frame 3, and meantime the soft pads 42 touch the user softly. Notably, the lenses 5 are arranged slantwise in the left frame 2 and the right frame 3. In use, the lenses 5 fit to the user's face and eyes for maintaining appropriate angle relative to the eyes and close to the eyes, thereby reducing resistance when jumping into water and enlarging visual field of the user. The supporting portions 27, 37 properly support the nose support 40 of the connecting frame 4 for providing comfortable touch and avoiding deformation of the connecting frame 4.

Generally speaking, according to features of the present invention, in order to remain lenses to align with the user's eyes, the width between upper edges of the lenses and upper edges of the left and the right frames is smaller than the width between lower edges of the lenses and lower edges of the left and the right frames. At the same time, the lenses are slantwise assembled in the left and the right frames. Preferably, a plurality of feeding holes are defined at rims of lenses and are unitarily formed with the left and the right frames when the left and the right frames are shaped.

It is understood that the invention may be embodied in other forms without departing from the spirit thereof. Thus, the present examples and embodiments are to be considered in all respects as illustrative and not restrictive, and the invention is not to be limited to the details given herein.

1. Swimming goggles comprising:
a left frame and a right frame being made of hard material, and respectively including an inner surfaces, an outer surfaces and a peripheral surfaces between the outer surfaces and the inner surfaces, an enveloping frames being respectively formed around the peripheral surfaces and slightly offsetting relative to outer peripheries of the inner surfaces, a lenses being unitarily formed with outer surfaces, width between upper edges of inner surface of the lenses and upper edges of the enveloping frames being smaller than width between lower edges of inner surface of the lenses and lower edges of the enveloping frames, a fastener bases being respectively formed on outward sides of the enveloping frames and defining a body and a fastener holes in body thereof;
head strap being respectively movably received in the fastener holes of the fastener bases; and
a connecting frame being made of soft material and connecting with the left frame and the right frame together, the connecting frame including a nose support, and a linking portion integrally extending from the nose support for encircling the enveloping frames of the left frame and the right frame.

2. The swimming goggles as claimed in claim 1, wherein the body of each of the fastener base space a distance from the inner surfaces of the left frame and the right frame, thereby providing more space for the connecting frame to support the left frame and the right frame.

3. The swimming goggles as claimed in claim 2, further comprising a connecting portions are respectively formed between the fastener base and the enveloping frame for spacing the bases of the fastener bases from the inner surfaces of the left frame and the right frame, thereby facilitating the connecting frame to support the left frame and the right frame.

4. The swimming goggles as claimed in claim 3, further comprising a projections are formed around outer peripheries of the enveloping frames for providing more space for the connecting frame to support the left frame and the right frame.

5. The swimming goggles as claimed in claim 4, further comprising a support portions integrally and inwardly extend from the enveloping frames.

6. The swimming goggles as claimed in claim 5, wherein the support portions has a through holes for facilitating the connecting frame to support the left frame and the right frame.

7. The swimming goggles as claimed in claim 1, wherein the connecting frame further includes pads connecting with the linking portion for providing comfortable touch for a user

8. The swimming goggles as claimed in claim 1, wherein the width between upper edges of inner surface of the lenses and upper edges of the enveloping frames meet along upper edge of inside of user's eyehole, and the width between lower edges of inner surface of the lenses and lower edges of the enveloping frames meet along lower edges of inside of user's eyehole.

9. Swimming goggles comprising:
a left frame and a right frame respectively including an inner surfaces, an outer surfaces and a peripheral surfaces between the outer surfaces and the inner surfaces, a lenses being formed between the outer surfaces and the inner surfaces;
a nose support being formed between the left frame and the right frame; and
head strap being respectively assembled at outer sides of the left frame and the right frame;
wherein the lenses are slantwise arranged in the left and the right frames, and the width between upper edges of inner surface of the lenses and upper edges of the left and the right frames is smaller than the width between lower edges of inner surface of the lenses and lower edges of the left and the right frames.

10. The swimming goggles as claimed in claim 9, wherein the lenses are unitarily formed with the left frame and the right frame.

11. The swimming goggles as claimed in claim 10, wherein a plurality of feeding holes are defined at rims of lenses and are unitarily formed with the left and the right frames when the left and the right frames are shaped.

12. The swimming goggles as claimed in claim 9, wherein the width between upper edges of inner surface of the lenses and upper edges of the enveloping frames meet along upper edge of inside of user's eyehole, and the width between lower edges of inner surface of the lenses and lower edges of the enveloping frames meet along lower edges of inside of user's eyehole.
